(19) United States
(12) Patent Application Publication
(10) Pub. No.: US 2007/0060497 A1
(43) Pub. Date: Mar. 15, 2007

(54) Reagents for modifying biopharmaceuticals, the use and production thereof

(76) Inventors: Ralf Krahmer, Berlin (DE); Frank Leenders, Berlin (DE)

Correspondence Address: FITCH EVEN TABIN AND FLANNERY, 120 SOUTH LA SALLE STREET, SUITE 1600, CHICAGO, IL, 60603-3406, US

(21) Appl. No.: 10/559,996

(22) PCT Filed: Jun. 11, 2004
(86) PCT No.: PCT/EP04/06315

§ 371 Date: Oct. 4, 2006

(30) Foreign Application Priority Data
Jun. 11, 2003 (DE) 10326303.9

(51) Int. Cl. A61K 38/17; C40B 40/04; C40B 40/10; C40B 30/06; C07C 229/02; C07C 237/02

(52) U.S. Cl. 514/2; 435/7.1; 530/409; 564/152; 560/19; 560/155

(57) ABSTRACT

The present invention relates to compounds which are suitable for coupling to pharmaceuticals, in particular biopharmaceuticals, and to conjugates composed of the compounds and biomolecules or pharmaceutical active compounds. The compounds according to the invention can be readily formed by means of multicomponent reactions. The invention also relates to the use of the conjugates as an improved formulation of pharmaceuticals, and to their preparation. The invention furthermore provides a laboratory kit for the in-vitro preparation of conjugates which are composed of the compounds according to the invention and pharmaceuticals as well as biotechnological substances, in particular biopharmaceuticals, pharmaceutical active compounds, synthetic molecules or surfaces.

The present invention relates to compounds which are suitable for coupling to pharmaceuticals, in particular biopharmaceuticals, and to conjugates composed of the compounds and biomolecules or pharmaceutical active compounds. The compounds according to the invention can be readily formed by means of multicomponent reactions. The invention also relates to the use of the conjugates as an improved formulation of pharmaceuticals, and to their preparation. The invention furthermore provides a laboratory kit for the in-vitro preparation of conjugates which are composed of the compounds according to the invention and pharmaceuticals as well as biotechnological substances, in particular biopharmaceuticals, pharmaceutical active compounds, synthetic molecules or surfaces.

The development of biopharmaceuticals as medicaments, or for potential medicaments, and of biotechnological products for use in the field of proteomics or in the industrial field has made rapid advances in recent decades, with these advances having been crucially influenced by several factors:
a) improved isolation and purification techniques,
b) the revolutionary developments in genetic engineering and, associated with these developments, the possibility of preparing recombinant proteins,
c) the improved understanding of biochemistry and of the mechanisms of action of biopharmaceuticals, and
d) the opening up of new areas of application and methods for biotechnological products.

The efficacy and the duration of the effect of an active compound are determined by its pharmacological profile. A rapid loss of activity, which, in a general manner, is termed “clearance”, is very frequently observed in vivo in the case of biopharmaceuticals, in particular. The clearance takes place as a result of processes such as metabolism and renal excretion and as a result of the reaction of the immune system on the exogenous compound. Particularly proteinogenic active compounds, which constitute an important group of biopharmaceuticals, elicit a powerful immune response when being used therapeutically, with this response being able to lead to allergic shock. In many cases, such disadvantageous effects prevent this otherwise very advantageous class of active compounds from being used commercially or therapeutically.

Nevertheless, scientists have for many years been engaged in developing strategies for enabling biopharmaceuticals to be used therapeutically. One of the first methods was that of changing the surface charge by reacting proteins with succinic anhydride. This modification is termed succinylation (Habeeb, A.F.S.A. Arch. Biochem. Biophys. 1968, 121, 652). Covalently bonding a biologically active compound to a very wide variety of polymers constitutes one of the most successful strategies in recent years and has become one of the most important methods for improving the pharmacological and toxicological properties of biopharmaceutical active compounds. One of the polymers which is most frequently employed in this connection is the polyalkylene oxide polyethylene glycol, termed PEG for short.

Abuchowski, one of the pioneers in the field of the polymer-mediated administration of biopharmaceuticals, showed that covalently coupling polyethylene glycol chains to a polypeptide molecule generates a positive pharmacological effect in the case of this active compound. The immunogenicity of a conjugate of this nature is reduced, while its half-life in the blood is prolonged (U.S. Pat. No. 4,179,337, Davis et al.; Abuchowski & Davis “Enzymes as Drugs”, Holcenberg & Roberts, Eds. John Wiley & Sons, N.Y. 1981, 367-383). Furthermore, modifying biotechnological products, such as enzymes, frequently influences other biochemical parameters such as their pH stability and thermostability. A modification can therefore, because of an increase in thermostability, be advantageous, for example, for industrial enzymes which are to be used in washing agents and, because of an increase in pH stability, be advantageous for biopharmaceuticals with regard to the latter being administered orally.

The above-described studies greatly accelerated research in the field of the conjugation of active compounds with the polymer polyethylene glycol. The modification with polyethylene glycol also offers some advantages in the case of small conventional active compounds. The covalent bonding of a small active compound to the hydrophilic molecule polyethylene glycol increases the solubility of the conjugate and can also reduce toxicity (Kratz, F. et al. Bioorganic & Medicinal Chemistry 1999, 7, 2517-2524). The most important reviews on conjugation with polyethylene glycol are the following: Greenwald, R. B., J. of Controlled Release 2001, 74, 159-171; Zalipsky, S. Advanced Drug Delivery Reviews, 1995, 16, 157-182; Zalipsky, S. Bioconjugate Chem. 1995, 6, 150-165; Jain, N. K.; et al. Pharmazie 2002, 57, 5-29.

The chemical reaction for coupling a polyethylene glycol molecule to a biopharmaceutical requires one of the two components which are involved in the reaction to be activated. As a rule, the PEG molecule is, for this purpose, provided with a connecting molecule, i.e. what is termed the activated linker. The whole spectrum of long established peptide chemistry is available for the activation. For the purpose of modifying amino functionalities, usually belonging to lysine residues, as building blocks of a biologically active compound, the linker is frequently activated in the form of an N-hydroxysuccinimide active ester. Harris, J. M. et al. (U.S. Pat. No. 5,672,662) developed this method for propionic acid and butyric acid linkers, while, in the case of Zalipsky, S. et al. (U.S. Pat. No. 5,122,614), an activated carbonic ester is employed. The reaction of a lysine residue with such an activated linker leads to the formation of an amide bond or urethane bond. The linking of a PEG to a biopharmaceutical is termed PEGylation, with this leading, in a number of cases, to loss of the biological activity. A reason for this can be the loss of the positive charge as a result of the formation of an amide bond at the lysine residue.

Reductive amination using PEG aldehydes represents a good alternative to that of using active esters (Harris, J. M. U.S. Pat. No. 5,252,714) because this coupling method leads to the formation of a secondary amine with the positive charge being preserved. Other coupling possibilities consist in using the maleimide method (cysteine residues) and in direct linkage, without any linker group, when using tresyl or halogen compounds.

The most frequently employed PEGs are linear monomethoxypolyethylene glycol chains (m-PEGs). These linear chains are not restricted conformationally and can rotate freely depending on the environment. Consequently, the surface of the biopharmaceutical which is shielded by the chains is relatively small. Branched modifying reagents, which contain several PEG chains in one molecule, are being developed for the purpose of improving the surface shielding. There are only a few commercial examples of this substance class. A known example of this class is an activated lysine which is provided with two m-PEG chains. However, because the bonds are freely rotatable in this case as well, the shielding effect is only moderate (Veronese, F. M. Bioconjugate Chem. 1995, 6, 62-69).

Even though PEGylation has already been developed very extensively, some crucial disadvantages still remain:
a) in many cases, modifying biopharmaceuticals leads to a dramatic decline in biological activity,
b) for process reasons, polymers such as polyethylene glycol exist as complex mixtures of different molecular weights, with this being termed polydispersity and frequently leading to problems in regard to reproducibility or quality,
c) depending on the quality of the m-PEG and the nature of the activation, undesirable crosslinking reactions occur in some cases,
d) optimizing the reaction conditions, assessing the pharmacological effect and selecting the correct modifying reagent are difficult and time-consuming, and
e) modifying biopharmaceuticals with polymers such as polyethylene glycol has thus far been the preserve of specialist laboratories.

Because of the above-described deficiencies in the prior art, there is a great need for modifying reagents which possess novel, variable properties and whose use results in crucial improvements in biotechnological products and in conventional synthetic products. It would furthermore be desirable to also make this technology available to users who do not have their own special laboratory equipment at their disposal or do not have any access to specialist laboratories.

An object of the invention was therefore to providers compounds which can be bonded to biopharmaceuticals and using which the disadvantages of biopharmaceutical conjugates of the prior art can at least partially be overcome. Another object was to provide a laboratory kit which enables any inclined scientist to modify a substance with polymers, such as polyethylene glycol.

According to the invention, this object is achieved by providing compounds of the formula (Ia/b) 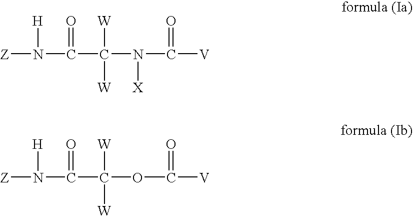
where compounds of the formula (Ia) can be prepared by means of a Ugi reaction and compounds of the formula (Ib) can be prepared by means of a Passerini reaction, and
in which
the residues V, W, X and Z are in each case, independently of each other, a hydrocarbon residue which can contain heteroatoms and/or V, W and/or X is/are hydrogen, characterized in that at least one of the residues V, W, X and/or Z carries a binding group Y and in that the residues V, W, X and Z together exhibit at least one group of the formula (II) 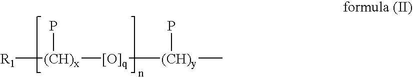
in which
P is, on each occasion independently, H, OH, C₁-C₄-alkyl, O—R₂ or CO—R₃,
R₁ is H, OH or a hydrocarbon residue which has from 1 to 50 carbon atoms and which can contain heteroatoms, in particular O and/or N,
R₂ is, on each occasion independently, a hydrocarbon residue having from 1 to 6 C atoms,
R₃ is OH or NR₄R₅,
R₄ and R₅ are, in each case independently, H or a hydrocarbon residue which can contain heteroatoms, in particular O and/or N, where R₄ and R₅ can also together form a ring system,
n is, on each occasion independently, an integer of from 1 to 1000, and
x is, on each occasion, an integer of from 1 to 10, and
y is an integer of from 0 to 50, and
q is, on each occasion independently, 0 or 1.

The compounds according to the invention exhibit a skeletal structure which can be obtained by means of a multicomponent reaction, for example a Ugi reaction or a Passerini reaction, or by means of a Ugi reaction which is carried out stepwise. In a Ugi reaction which is carried out stepwise, three components (amine component, isonitrile component and carbonyl component) are initially reacted with each other and the fourth component (acid component) is then coupled to the reaction product. Using such a multicomponent reaction makes it possible, when selecting suitable starting compounds, to selectively prepare functional groups in a molecule in a simple manner. The compounds according to the invention contain, as the functional group, at least one binding group Y which enables the compound according to the invention to be bonded covalently to other molecules, in particular to biotechnological, pharmaceutical or synthetic active compounds, and also to surfaces or biocatalysts. The binding group Y is preferably a compound which can bind covalently to a functional group which is present in the active compound to be coupled, for example a binding group which is able to bind to an amino group, a thiol group, a carboxyl group, a guanidine group, a carbonyl group, a hydroxyl group, a heterocycle, in particular containing N as the heteroatom (e.g. in histidine residues), a C-nucleophilic group, a C-electrophilic group, a phosphate, a sulfate or similar. Noncovalent bonds, e.g. chelates, complexes with metals, e.g. at surfaces or with radioisotopes, as well as bonds to silicon-containing surfaces, are also possible. Examples of suitable binding groups are a carboxylic acid or an activated carboxylic acid group.

For subsequent coupling of the compound to a biotechnological or synthetic product as well as to natural products and technical products, the compounds according to the invention preferably contain an activated functionality Y. In the activated form, Y is preferably selected from the group consisting of (O-alkyl)₂, —OSO₂CH₂CF₃ (tresyl), (O-aryl)-azides, —CO-Q, maleimidyl, —O—CO-nitrophenyl or trichlorophenyl, —S—S-alkyl, —S—S-aryl, —SO₂-alkenyl (vinylsulfone), or -halogen (Cl, Br or I), where Q is selected independently from the group consisting of H, O-aryl, O-benzyl, O—N-succinimide, O—N-sulfosuccinimide, O—N-phthalimide, O—N-glutarimide, O—N-tetrahydrophthalimide, N-norbornene-2,3-dicarboximide, hydroxybenzotriazoles and hydroxy-7-azabenzotriazoles. Y is preferably a —CO-Q group. The review by Zalipsky, S., which appeared in Bioconjugate Chem. 1995, 6, 150-165, provides a good overview of possible activations.

The group Y enables the compounds according to the invention to be bonded covalently to active compounds, thereby forming highly desirable, stable conjugates.

The compounds according to the invention furthermore exhibit at least one group of the formula (II). The compounds preferably exhibit at least two, and even more preferably three, groups of the formula (II). Due to the flexibility provided by the multicomponent reaction, it is possible to insert the groups of the formula (II) at different positions in the molecule. Thus, it is possible to insert groups of the formula (II) into different residues V, W, X and/or Z, in particular into X and/or Z. In this way, it is possible to prepare a compound which contains several, and in particular a large number, of groups of the formula (II) which, in particular, confer a reduced immunogenicity, a prolonged half-life in the body, a higher proteolysis stability, an increase in solubility, a reduction in toxicity, an improved pH stability and an improved thermostability on a conjugate which is composed of a compound of the formula (I) and an active compound.

Alternatively, or in addition, it is also possible to insert several groups of the formula (II), preferably two groups of the formula (II), into one of the residues V, W, X and/or Z, in particular X and/or Z.

In particular, it is possible, according to the invention, to achieve good shielding using one or more short-chain groups of the formula (II), with it being possible to obtain and introduce, with good reproducibility, short-chain groups of the formula (II) having the same chain length. Alternatively, it is also possible to simultaneously introduce groups of the formula (II) having different chain lengths. It is furthermore also possible to employ polydisperse groups of the formula (II).

It was found, in accordance with the invention, that good shielding of active compounds which are coupled to compounds according to the invention can already be achieved when the compounds of the formula (I) according to the invention exhibit a molecular weight of from 200 to 50 000 Da, in particular of from 1000 to 20 000 Da. It was furthermore found that compounds of the formula (I) according to the invention which contain more than one chain of the formula (II) already bring about good shielding at lower molecular weights of the total compound. In the case of compounds which exhibit two to three groups of the formula (II), a molecular weight of the total compounds of from 500 to 25 000 Da, in particular of from 500 to 10 000 Da, is already sufficient. Compounds which exhibit four or five groups of the formula (II) preferably have a molecular weight of from 200 to 12 500 Da, in particular of from 500 to 7500 Da. In the case of compounds which contain six or more groups of the formula (II), the molecular weight is particularly preferably ≦7500 Da, and even more preferably ≦5000 Da.

The groups of the formula (II) are preferably polyalkylene oxides, such as polyethylene glycol, polyolefin alcohols such as polyvinyl alcohol, or polyacryl morpholine.

In the groups of the formulae (II), the residues or spacers P, R₂, R₃, R₄, R₅, n, x, y and q can, in a molecule or a residue, in each case be identical or else, independently of each other, different. Thus, the residue of the formula (II) can, for example, be a polyalkylene oxide which is composed of polyethylene oxide groups and polypropylene oxide groups.

When P═CO—R₃, the groups are polyacrylic acid groups (R₃═OH) or polyacrylamides (R₃═NR₄R₅). In this connection, R₄ and R₅ can be hydrogen or a hydrocarbon residue having from 1 to 30 C atoms, in particular from 1 to 10 C atoms, more preferably from 1 to 6 C atoms, which residue can contain heteroatoms, in particular one or more heteroatoms which are selected from O, N, P and S. The residues R₄ and R₅ can also together form a ring, for example a morpholine ring.

The residue R₁ is hydrogen, hydroxyl or a hydrocarbon residue having from 1 to 50 carbon atoms, more preferably from 1 to 30 carbon atoms and most preferably from 1 to 10 carbon atoms, which residue can optionally contain heteroatoms, in particular O, N, S, P and/or Si. The residue R₁ can be saturated or singly or multiply unsaturated, and also be linear, branched or cyclic. Particularly preferably, R₁ is HO, CH₃—O, CH₃—(CH₂)ₐ—O or (CH₃)₂CH—O, where a is an integer between 1 and 20. R₁ can also preferably be selected from an acetal, e.g. (CH₃O)₂— and (CH₃—CH₂O)₂—, an aldehyde, e.g. OHC—CH₂—O—, an alkenyl group, e.g. CH₂═CH—CH₂—O—, an acrylate, e.g. CH₂═CH—CO₂—, or a methacrylate, e.g. CH₂═C(CH₃)—CO₂—, an acrylamide, e.g. CH₂═CH—CONH—, an aminoalkyl group, e.g. H₂N—CH₂—CH₂—, a protected aminoalkyl group, e.g. A-NH—CH₂—CH₂—, where A is a protecting group, in particular N-acyl, N-sulfonyl or N-silyl protecting groups, such as tert-Boc-, Alloc-, Fmoc, Tr-, Z- or Moz-, a thioalkyl group HS—CH₂—CH₂— or a protected thioalkyl group.

The group of the formula (II) preferably has the formula (IIa)
R₁—(CH₂—CH₂—O)ₙ—CH₂—CH₂— formula (IIa)
where n is between 0 and 1000.

n (as used herein, e.g. in formula II or formula IIa) is, on each occasion independently, an integer of from 0 to 1000, more preferably of from 1 to 500, even more preferably of from 2 to 250, in particular at least 3 and most preferably from at least 4 to 50. According to the invention, it is possible to prepare compounds having a large number of groups of the formula (II), preferably having at least 2, in particular at least 3, preferably at least 4, more preferably at least 5, and most preferably at least 9, groups of the formula (II).

Frequently, however, compounds which contain 2 or 3 groups of the formula (II) are already particularly preferred.

x is, on each occasion independently, an integer of from 1 to 10, in particular of from 1 to 6, more preferably of from 2 to 3, and y is an integer of from 0 to 50, more preferably of from 1 to 10, even more preferably of from 2 to 6. q is, on each occasion independently of each other, 0 or 1.

The residues V, W, X and Z are derived from the starting compounds which are reacted in the multicomponent reaction or, when one of the starting compounds possesses two or more functional groups (amine, ketone, aldehyde, isonitrile or acid group) are synthesized during the course of the multicomponent reaction. Preference is given to compounds which are obtained in a multicomponent reaction or a multi-step multicomponent reaction, in particular a four-component reaction and most preferably in a Ugi reaction, in which at least one starting compound which is branched, i.e. possesses at least two, more preferably at least three, groups (e.g. amine, carbonyl, isonitrile and/or acid group) which are reactive in the multicomponent reaction, is employed.

When the compounds according to the invention are prepared using a Ugi reaction, the residue V is derived from the acid component, the residue Z is derived from the isonitrile component, the residue X is derived from the amino components and the residue W is derived from the carbonyl component.

The residues V, W, X and Z are, in each case independently of each other, hydrogen or a hydrocarbon residue which can optionally contain heteroatoms. In this present document, a hydrocarbon residue means, unless otherwise explicitly indicated, a residue having from 1 to 100 000 C atoms, more preferably a residue of from 1 to 10 000 C atoms, in some preferred cases from 1 to 50 C atoms, which residue can contain from 0 to 10 000, more preferably from 1 to 1000, heteroatoms, which are selected, for example, from O, P, N or S. The hydrocarbon residues can be linear or branched and be saturated or singly or multiply unsaturated. A hydrocarbon residue can also contain cyclic or aromatic segments. Preferred hydrocarbon residues are alkyl, cycloalkyl, alkenyl, cycloalkenyl, alkynyl, cycloalkynyl, aroyl and heteroaroyl. However, a hydrocarbon residue, as used herein, can also contain functional groups and, in particular, a targeting agent and can comprise, for example, an aminocarboxylic ester, for example a saturated or unsaturated omega-aminocarboxylic ester, a dye, a fluorescence label, an antibiotic, a minor or major groove binder, a biotinyl residue, a streptavidin residue, an intercalating residue, an alkylating residue, a steroid, a lipid, a polyamine, folic acid, a receptor agonist or receptor antagonist, an enzyme inhibitor, a peptide, an antibody or an antibody fragment, an amino sugar, a saccharide or oligosaccharide, e.g. galactose, glucose or mannose, an antisense polymer, a modified surface, a surface-active agent or a complexing agent.

In a preferred embodiment, at least one of the residues V, W, X and/or Z comprises a targeting group which enables the compounds according to the invention and, in particular, conjugates containing the compounds according to the invention, to be directed selectively to a desired target site, for example a site of a disease, such as a focus of inflammation or a cancer tumor. Folate, biotin, mannose, maltose, succinate, aconitate, dexamethasone, alkylglycosides, glycosides and peptides, e.g. with an Arg-Gly-Asp motif, can, for example, serve as targeting groups.

According to the invention, it is also possible to prepare molecules which contain two or more targeting groups. This thereby makes it possible to achieve an increased targeting effect and/or to direct the compound, or a conjugate which is formed therewith, to several desired sites.

Furthermore, the compounds according to the invention can also contain reporter groups, for example fluorescent dyes or fluorescent labels, which permit use for diagnostic purposes.

The residue X in the compounds according to the invention (the residue which is introduced, in a Ugi reaction, by using a primary amine X—NH₂) is preferably a targeting group, a residue of the formula (II), or a combination of the two. In this connection, x particularly preferably =2, 3 or 4. Ethylene glycol, propylene glycol, butylene glycol, or combinations thereof having a chain length of from 3 to 500, in particular of from 4 to 100, units, are particularly preferred subunits in the residue X. R₁ in the residue X is particularly preferably methoxy or ethoxy, in particular methoxy. Most preferably, X is methoxypolyethylene glycol having from 1 to 1000, in particular from 4 to 50, ethylene units. Short-chain methoxypolyethylene glycol residues, for example having 3 to 10, in particular having 3 to 4, ethylene units are particularly preferably employed in monodisperse form. In another preferred embodiment, the residue X contains a targeting group as specified above. In a particularly preferred embodiment, a residue X contains the shielding function, as a result of the formula (II), and the targeting function. Such a residue X preferably has the formula (IIb) 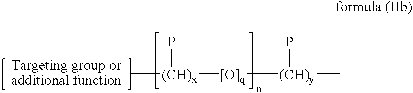
in which the meanings of the spacers in this formula are as specified above.

The residue Z, which is derived from the isonitrile (Z-NC) when the compounds according to the invention are prepared using the Ugi reaction, is preferably a C₁-C₈-alkyl residue or a residue which contains one, two or more groups of the formula (II) as well as, where appropriate, a targeting function. Z is particularly preferably a group of the formula (Xa), (Xb) or (Xc) 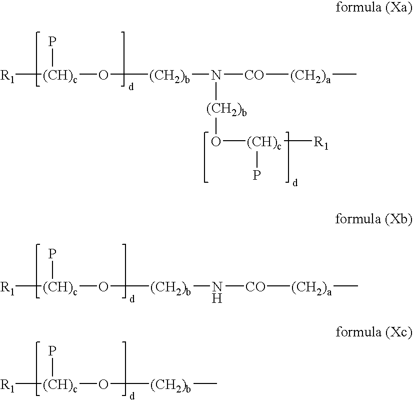
and, in particular, 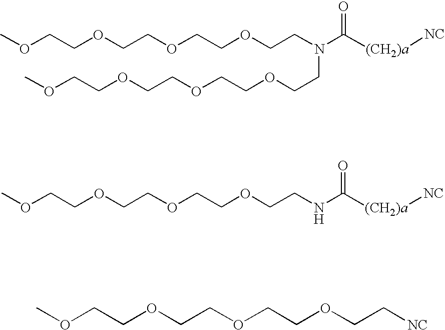
in which
P is, on each occasion independently, H, OH, C₁-C₄-alkyl, O—R₂ or CO—R₃ (where R₂ and R₃ are defined as above),
R₁ is H, OH or a hydrocarbon residue which has from 1 to 50 carbon atoms and which can contain heteroatoms and is preferably a C₁-C₁₀-alkoxy residue,
a is, on each occasion, an integer of from 0 to 50, in particular of from 1 to 3,
b is, on each occasion, an integer of from 0 to 50, in particular of from 1 to 3,
c is, on each occasion, an integer of from 1 to 10, in particular of from 2 to 4, and
d is, on each occasion independently, an integer of from 1 to 1000, in particular of from 5 to 100.

The residues W, which are derived from the carbonyl compound when the compounds according to the invention are prepared by means of a Ugi reaction, are, on each occasion independently, preferably hydrogen or a C₁-C₆-hydrocarbon residue, in particular a C₁-C₄-alkyl residue, and most preferably hydrogen, methyl or ethyl. In a particularly preferred embodiment, the two residues W in compounds of the formula (I) are identical and are consequently derived from formaldehyde (W═W═H) or from symmetrical ketones such as acetone or 3-pentanone. Using symmetrical ketones prevents the formation of a center of symmetry at the carbon atom to which the residues W are bonded. As a result, no problems associated with chiral compounds arise when forming conjugates with active compounds. W is particularly preferably on each occasion hydrogen.

In another preferred embodiment, the residue W is introduced by using an aldehyde as starting compound in the Ugi reaction. In this case, one of the W residues is hydrogen while the other W residue is preferably a C₁-C₆-hydrocarbon and, in particular, a C₁-C₄-alkyl residue. In this case, one of the W residues can contain a group of the formula (II), a linker and/or a targeting group.

Finally, the residue V is derived from the carboxylic acid compound when preparing the compounds according to the invention using a Ugi reaction. The group V preferably contains a linker or a binding group Y for coupling the compounds according to the invention to other molecules, in particular to biotechnological, pharmaceutical or synthetic active compounds. In addition to the binding group, the residue V can contain a linker group, preferably a C₁-C₈-alkylene group or a glycol group, for example a tetraethylene glycol group.

In the above-described preferred embodiment, the compounds of the formula (I) preferably possess one to three, more preferably two to three, groups of the formula (II), namely a group in the residue X and one or two groups in the residue Z.

A particularly preferred structure of these compounds is shown below as formula (XI), where n=0 to 10.

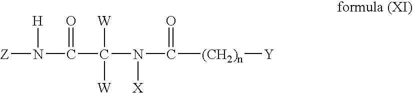

While one preferred embodiment of the invention, namely that of preparing compounds by means of a Ugi reaction using monofunctional starting materials, was explained in more detail above, polyfunctional starting materials can be employed in another embodiment which is preferred in accordance with the invention. For this, at least one of the starting materials is employed in the Ugi reaction in polyfunctional form, that is in bifunctional, trifunctional or higher-functional form. Particular preference is given to using at least one bifunctional starting material, that is a dicarboxylic acid, a diamine, a diisonitrile and/or a dialdehyde or diketone, and preferably at least one dicarboxylic acid and/or one diamine. Using such polyfunctional starting materials results in compounds of the formula (I) in which several groups V, X, W and Z and, in particular, several groups X and Z, are present and consequently a large number of groups of the formula (II) can be envisaged. An example of these compounds, in which a tricarboxylic acid was used as the starting material, is represented by the following general formula (III): 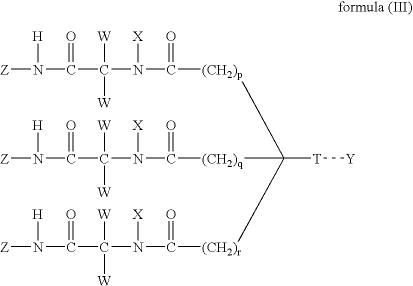
and, in particular, 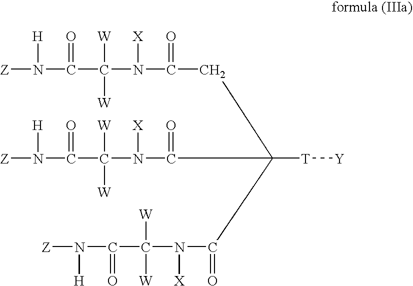
where
p, q and r can, independently of each other, be an integer between 0 and 50, more preferably between 0 and 10. r is preferably=0.

Compounds of formula (III) can be prepared using a process which is based on a Ugi 4-component reaction in which a carbonyl component, an amino component, an isonitrile component and an acid component participate. These components can, where appropriate, be reacted with each other simultaneously and contain protecting groups which are subsequently removed or which remain in the molecule.

The acid component in formula (IIIa) is in this case a 1,1,2-ethanetricarboxylic acid which additionally carries a linker group at the 1 position. The carbonyl component which is used for preparing compounds of the formula (IIIa) is preferably formaldehyde or a symmetrical carbonyl compound, e.g. acetone or cyclohexanone. This thereby avoids the formation of diastereoisomeric mixtures. It is alternatively also possible to use asymmetric aldehydes, e.g. isobutyraldehyde, or ketones.

The linker T is preferably represented by an alkyl chain which is branched or unbranched, saturated or unsaturated and can contain heteroatoms, in particular N, S and O, for example between the branching and T. T preferably possesses a carbon atom or a nitrogen atom as the linkage to the branching site in the compounds of formula (III) or (IIIa). More preferably T is an alkyl chain of the structure 1.
T=—(CH₂)ₘ— structure 1
where m is an integer of from 1 to 10, preferably, however, an integer of from 1 to 5.

When

Y is an acetal, the linker has the structure

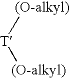

Other preferred compounds in which a dicarboxylic acid was used as starting material are represented by the general formula (XII): 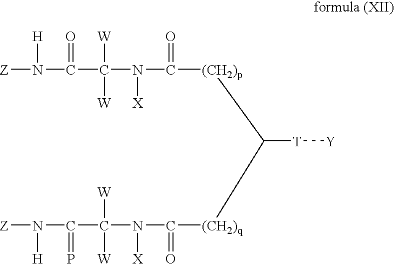
in which p and q are in each case integers of from 0 to 5. Preferred compounds which can be obtained by using diamines are depicted by the general formula (XIII): 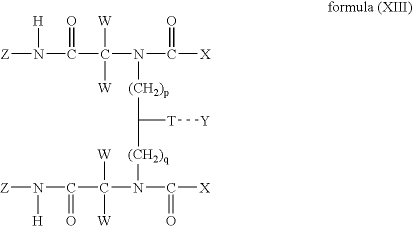
in which p and q are in each case integers of from 0 to 5.

The present invention contributes to reducing the disadvantages and restrictions which have been described and which occur in the prior art. It encompasses the synthesis of bifunctional compounds which can be used for modifying natural products, industrial products, biotechnological and synthetic products or pharmaceutical active compounds.

The compounds according to the invention contain an activated linker group, which enters, within the context of a chemical reaction under mild reaction conditions, into a covalent bond with one or more amino functionalities or other functional groups of a biotechnological or synthetic product, and at least one polymer function which influence the biochemical and pharmacological properties of the conjugate. In preferred embodiments, the compounds contain additional functions such as targeting functions.

The present invention provides what is preferably a multibranched structure, as well as its synthesis and use for modifying biotechnological products. The structure can be prepared using a multicomponent reaction, e.g. the Ugi reaction (Ugi, I. et al., Angew. Chem. Int. Ed. 2000, 39, 3168-3210: EP 1104677). The use of the multicomponent reaction makes it possible to take a combinatorial approach and also enables the preparation to be automated.

The present invention preferably provides an unbranched or branched polymer compound which carries only one single activated linker group, thereby avoiding crosslinking reactions. This polymer compound is hydrophilic and biologically tolerated. It is simple to prepare and opens up broad possibilities of application in connection with modifying pharmaceutical active compounds and products which are employed industrially. Conjugates of the polymer compound according to the invention and pharmaceutical active compounds enable therapeutic employment to be improved. Furthermore, by prolonging the duration of the effect, these conjugates make it possible to reduce the quantity of active compound to be administered as, for example, in the case of treating cancer diseases and infectious diseases.

The invention furthermore relates to a process for preparing the compounds according to the invention, where the individual components of the formulae
X′—NH₂ (IV)
(W′)₂C═O (V)
Z′-NC (VI)
and
V′—COOH (VII)
are reacted with each other in a multicomponent reaction, where V′, W′, X′ and Z′ are, in each case independently of each other, a hydrocarbon residue which can optionally contain heteroatoms and/or V′, W′ and/or X′ are hydrogen, where at least one of the residues V′, W′, X′ and Z′ carries a binding group Y and where the residues V′, W′, X′ and Z′ together possess at least one, in particular at least two, groups of the formula (II) 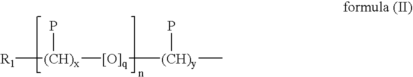
in which
P is, on each occasion independently, H, OH, O—R₂ or CO—R₃,
R₁ is H or a hydrocarbon residue which has from 1 to 50 carbon atoms and which can contain heteroatoms, in particular O, N, S, P and/or Si,
R₂ is, on each occasion independently, a hydrocarbon residue having from 1 to 6 C atoms,
R₃ is OH or NR₄R₅,
R₄ and R₅ are, in each case independently, H or a hydrocarbon residue which can contain heteroatoms, in particular O, N, S and/or P, where R₄ and R₅ can together also form a ring system,
n is, on each occasion independently, an integer of from 1 to 1000, and
x is, on each occasion, an integer of from 1 to 10, and
y is an integer of from 0 to 50, and
q is, on each occasion independently, 0 or 1.

A four-component reaction, more preferably a Ugi reaction or Passerini reaction, and most preferably a Ugi reaction, is, in particular, employed as the multicomponent reaction. When the residues X′, W′, Z′ and V′ do not exhibit any further functionality which is reactive for the multicomponent reaction (that is NH₂, CO, NC or COOH), the residues V′, W′, X′ and Z′ which are present in the starting compounds correspond precisely to the residues V, W, X and Z which can be found in the compounds according to the invention. Preference is given, however, to using at least one starting compound which contains an additional functionality (NH₂, CO, NC or COOH). In this case, a branched molecule is obtained. Examples of such starting compounds are 1,1,2-ethanetricarboxylic acid having three carboxylic acid residues, that is two carboxylic acid groups in the residue V′, or residues which contain at least two different functional groups, such as lysine (simultaneously contains an acid group and an amine group) or γ-aminobutyric acid. When such multifunctional starting compounds are used, the corresponding groups V, W, X and, respectively, Z in the product are only synthesized, starting from the functional group in the residue V′, W′, X′ and, respectively, Z′, in the multicomponent reaction. In this way, it is possible to synthesize highly branched and highly functional compounds, in particular compounds which contain a large number of groups of the formula (II), in a one-pot reaction.

In another preferred embodiment of the present invention, compounds which possess at least two groups of the formula (II) are prepared. These compounds have the general formula (XIV) 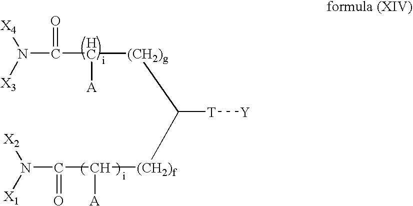
in which
h and i are, on each occasion independently, 0 or 1,
g and f are, on each occasion independently, an integer between 0 and 10, preferably between 0 and 5,
A is, on each occasion, H or —(CO)—NX₂, and
X₁, X₂, X₃ and X₄, and also X have, in each case independently of each other, the meanings given above for X.

T-Y is preferably the group —CH₂—CH₂—CH═CH₂, where any functionalities, for coupling to active compounds, can be inserted at the double bond.

Preference is furthermore given to compounds in which g=f, h=i, X₁═X₃ and X₂═X₄, with the carbon atom in the labeled position 1 not being a chiral center in these compounds. Achiral molecules which possess up to 6 (in the case of dicarboxylic acids) or up to 9 (in the case of tricarboxylic acids) groups of the formula (II) can be prepared, according to the invention, by linking a dicarboxylic acid or tricarboxylic acid to an amine which contains a group of the formula (II). Since, according to the invention, amines are coupled to dicarboxylic or tricarboxylic acids which are not amino acids, the coupling can be carried out simply without there being any necessity for an elaborate method of synthesis using protecting groups.

Within the context of the present invention, conjugates of the bifunctional, branched polymer compound with biologically active substances, such as proteins (e.g. human growth factors), enzymes, cofactors for enzymes (e.g. NAD+/NADH), liposomes, antibodies, small synthetic active compounds, phospholipids, lipids, nucleosides, oligonucleotides, microorganisms, human cells and surfaces are also prepared.

The invention therefore also relates to conjugates which comprise compounds of the formula (I) which are covalently linked to other molecules, in particular to active compounds, such as biopharmaceuticals or synthetic active compounds, or biotechnological substances which are employed in the “life science” field, e.g. in the field of proteomics or diagnostics. These substances are, for example, enzymes, in particular proteases, such as trypsin or chymotrypsin. The compounds which are linked, in the conjugates, to the compounds according to the invention are preferably biopharmaceuticals, active compounds of peptide nature or other biologically active substances. It is furthermore also possible for conjugates to be formed with surfaces or biocatalysts.

The invention furthermore relates to conjugates which comprise compounds of the formula (I) which are covalently linked to medicinal products or adjuvants for administering active compounds. By way of example, the linking-on of the compounds according to the invention enables tissues for heterotransplants, such as heart valves, to be made more readily tolerated by the recipient. Furthermore, adjuvants, such as liposomes or nanocapsules, for administering active compounds can be modified in order to confer on them desired properties, in particular a longer half-life in the body.

The invention furthermore relates to a pharmaceutical composition which comprises the compounds according to the invention and, in particular, the conjugates according to the invention. These pharmaceutical compositions can be employed, for example, for preventing or treating cancer, coronary diseases, metabolic diseases, neuronal or cerebral diseases or inflammatory processes, such as infections, immune diseases or autoimmune diseases (e.g. rheumatoid arthritis).

The compounds or conjugates according to the invention are also outstandingly suitable for being used as diagnostic agents.

Because of the reaction being multicomponent, it is readily possible, according to the invention, to prepare a great variety of compounds as claimed in claim 1. Varying the starting compounds makes it possible to obtain compounds which vary over wide ranges and which are matched to the given requirements. The present application consequently also relates to combinatorial libraries, or to the preparation of such libraries, which contain at least two, more preferably at least five, even more preferably at least 10, and most preferably at least 100, of the substances according to the invention. These libraries can be used to screen, in a simple manner, for the desired properties, for example ability to bind to active compound molecules or ability to shield particular active compounds, or for desired targeting properties.

Finally, it is readily possible, according to the invention, to provide a kit which comprises all the reagents and instructions, as well as the compounds according to the invention, which make it possible to modify proteins, nucleic acids or other active compounds, or else surfaces, with polymers in vitro in a simple manner. A substance is, for example, reacted with the compounds according to the invention such that the polymer compound according to the invention is added, at least in molar quantity based on the number of the modifiable reactive groups, e.g. amino groups (lysine residues, histidine, N terminus), carboxyl groups (aspartic acid, glutamic acid, C terminus), thiol groups (cysteine), hydroxyl groups (serine, threonine, tyrosine) or carbonyl groups (aldehydes), to a solution or a suspension of the substance to be modified, e.g. a protein, in aqueous buffer. The polymer compounds according to the invention are preferably employed in a molar excess of from 1 to 1000, more preferably in a molar excess of from 1 to 100, and particularly preferably in a molar excess of from 1 to 20, based on the modifiable groups.

Suitable reaction solutions are aqueous buffers such as from 0.001 to 1.0 molar solutions of sodium or potassium dihydrogen phosphate with disodium or dipotassium hydrogen phosphate or sodium, potassium or ammonium hydrogen carbonate with disodium, disodium or diammonium carbonate or tris(hydroxymethyl)aminoethane with hydrochloric acid; buffer solutions for the pH range between pH 4 and pH 10, particularly preferably between pH 5 and pH 9, are preferably suitable.

In the method according to the invention, the cosolvents methanol, ethanol, propanol, i-propanol, butanol, ethyl acetate, methyl acetate, dimethylformamide, acetonitrile, dimethyl sulfoxide or sulfolane can be added to the buffer in quantities of from 0.1 to 50% by vol., more preferably from 0.1 to 20% by vol., depending on the solubility of the coreactants. The reaction temperature is between 0° C. and 90° C., preferably from 4° C. to 40° C.

In addition, stabilizers or detergents, e.g. sodium azide, glycerol, ethylene glycols or ionic or nonionic detergents, can be added to the buffers.

In addition, the crude conjugate products which can be obtained using the method according to the invention can be purified by means of dialysis, chromatographic methods or ultrafiltration (including that for centrifuges) using aqueous buffer solutions or pure water, as well as by means of methods with which the skilled person is familiar, and then taken for their subsequent use.

Establishing the structure of the products (conjugates), i.e. analyzing the number of covalently bonded polymer compounds according to the invention, is effected by directly measuring the molecular weight, e.g. by means of MALDI-TOF mass spectrometry, by selectively determining one or more covalently bonded components or by indirectly detecting the unmodified groups. Thus, for example, a dye molecule which has been introduced by way of the compound according to the invention can be readily determined by measuring the extinction (UV/VIS). Furthermore, the number of unmodified amino groups can, for example, be determined fluorometrically by reacting with fluorescamine.

The stability of the conjugate towards proteases can, for example, be investigated as a direct demonstration of the improvement of the properties of the conjugate composed of a polymer compound according to the invention.

The invention is additionally explained by means of the attached examples and figures:

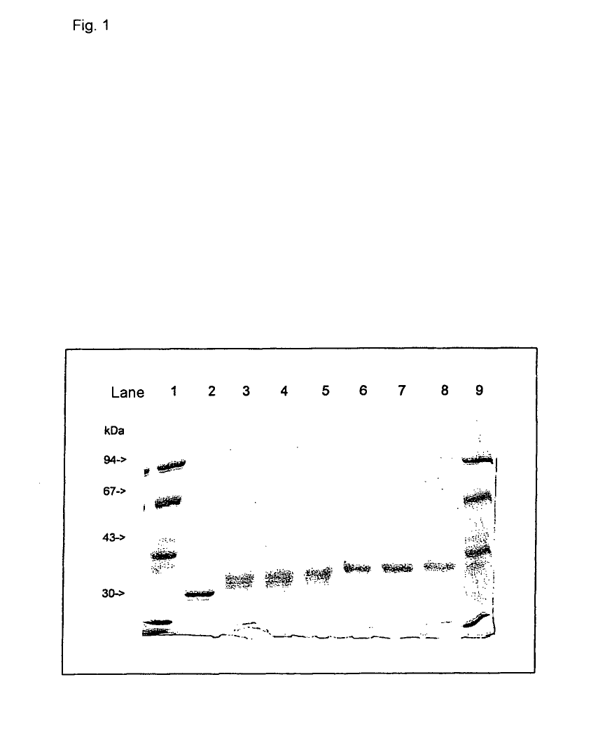

FIG. 1: SDS-PAGE analysis of conjugates composed of L-asparaginase and substance 16. The samples are: lanes 1) and 9) protein standard (low molecular weight markers, Amersham Pharmacia), lane 2) L-asparaginase (control, 2 μg), lane 3) modified L-asparaginase (0.5 eq. of substance 16), lane 4) modified L-asparaginase (1 eq. of substance 16), lane 5) modified L-asparaginase (2 eq. of substance 16), lane 6) modified L-asparaginase (5 eq. of substance 16), lane 7) modified L-asparaginase (10 eq. of substance 16) and lane 8) modified L-asparaginase (20 eq. of substance 16).

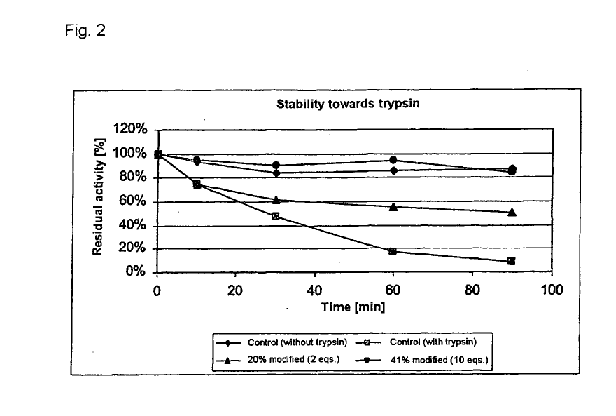

FIG. 2: Protease stability of a conjugate composed of L-asparaginase and substance 16:

Influence of the modification of L-asparaginase with substance 16 on the stability of L-asparaginase towards trypsin, as deduced from the residual activity. Modifying with substance 16 markedly increases the stability towards trypsin.

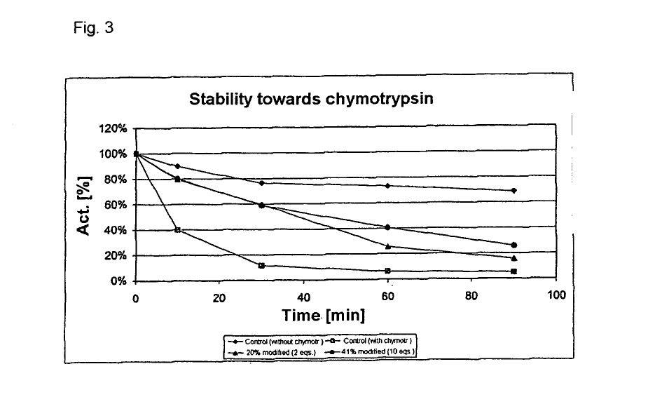

FIG. 3: Influence of the modification of L-asparaginase with substance 16 on the stability of L-asparaginase towards chymotrypsin, as deduced from the residual activity. Modifying with substance 16 markedly increases the stability towards chymotrypsin.

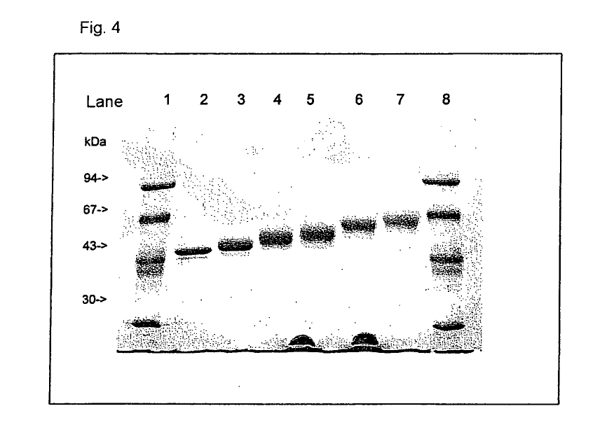

FIG. 4: SDS-PAGE analysis of conjugates composed of streptokinase and substance 16. The samples are: lanes 1) and 8) protein standard (low molecular weight markers, Amersham Pharmacia), lane 2) streptokinase (control, 2 μg), lane 3) modified streptokinase (0.5 eq. of substance 16), lane 4) modified streptokinase (1 eq. of substance 16), lane 5) modified streptokinase (2 eq. of substance 16), lane 6) modified streptokinase (5 eq. of substance 16) and lane 7) modified streptokinase (10 eq. of substance 16).

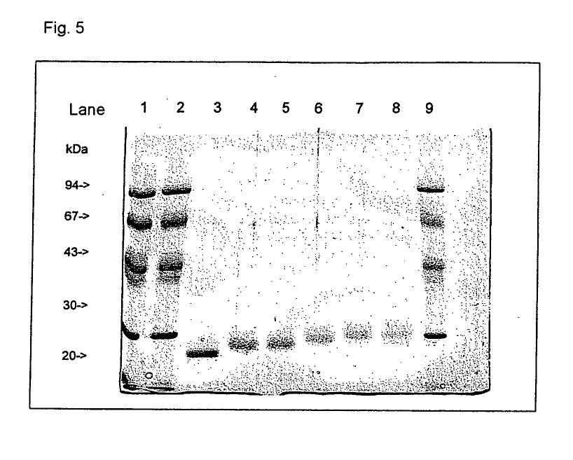

FIG. 5: SDS-PAGE analysis of conjugates composed of trypsin and substance 16. The samples are: lanes 1), 2) and 9) protein standard (low molecular weight markers, Amersham Pharmacia), lane 2) trypsin (control, 2 μg) lane 3) modified trypsin (0.5 eq. of substance 16), lane 4) modified trypsin (1 eq. of substance 16), lane 5) modified trypsin (2 eq. of substance 16), lane 6) modified trypsin (5 eq. of substance 16) and lane 7) modified trypsin (10 eq. of substance 16).

### A. EXAMPLES OF COMPOUNDS ACCORDING TO THE INVENTION AS CLAIMED IN CLAIMS 1 TO 6

In the context of the multicomponent reaction, an amino component, an oxo or carbonyl component, an isocyano component and an acid component are reacted to give the compound according to the invention.

The primary amines which are used can be obtained commercially or can be prepared from the monomethoxypolyethylene glycols by means of a Gabriel synthesis or from the corresponding azido compound by means of catalytic hydrogenation. Symmetrical or unsymmetrical secondary amines can be prepared from a primary amine by reductive amination using a corresponding aldehyde, which is obtained from monomethoxypolyethylene glycol by means of a Swern oxidation, for example, or can be obtained by means of simple substitution reactions.

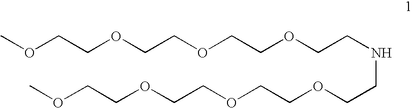

MS (ES+): m/z: 398.2 [M+H]⁺, 420.2 [M+Na]⁺; C₁₈H₃₉O₈.

A wide range of isonitriles can be obtained commercially. Furthermore, a large number of synthetic methods are available for preparing them. A very reliable method is that of preparing isonitriles from primary amines by reacting to give the formamide and subsequently dehydrating using phosgene or POCl₃ (I. Ugi; R. Meyr, Angew. Chem. 1958, 70, 702). Alternatively, isonitriles can be readily obtained by reacting a primary or secondary amine with a methyl or ethyl Ω-isocyanocarboxylate.

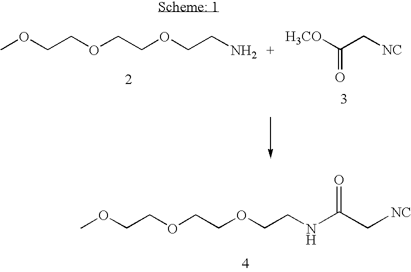

Methyl isocyanoacetate (1.82 g; 18.4 mmol) is added, at 20-25° C. and while stirring, to 2 (3.00 g; 18.4 mmol). The resulting reaction mixture is then stirred at 20-25° C. for 24 hours. Column-chromatographic purification yields 4 (3.64 g; 86%) as a pale yellow oil.

MS (ES−): m/z: 229.2 [M+H]⁻, MS (ES+) m/z: 231.1 [M+H]⁺; C₁₀H₁₈N₂O₄.

A large number of aldehydes or ketones can be used as the oxo or carbonyl component. In order, however, to avoid the formation of chiral centers and the enantiomer or diastereomer mixtures (higher degree of branching) which result therefrom, preference is given to using symmetrical ketones, such as acetone, and simple formaldehyde. There are a wide selection of synthetic possibilities for preparing aldehydes of polyethylene glycol or monomethoxyethylene glycol. They can be obtained by direct oxidation of the terminal hydroxyl function (e.g. Swern oxidation) or from unsaturated ethers or esters (e.g. allyl ethers) by oxidatively cleaving the double bond (e.g. ozonolysis, cat. OsO₄/NaIO₄).

The acid component simultaneously serves as the linker for the subsequent coupling to the active compound, which means that preference is given to using carboxylic acids which can be converted, by means of a few synthetic steps, after the multicomponent reaction has been completed, into an activated form of the compound according to the invention. These carboxylic acids can be monoesters of dicarboxylic acids (e.g. mono-tert-butyl succinate) or unsaturated monocarboxylic acids (e.g. 4-pentenecarboxylic acid). N-Substituted amino acids (e.g. N-Boc-L-glutamic acid, N-Boc-L-aspartic acid) or more highly branched carboxylic acids (e.g. tricarboxylic acid 7) can be used to achieve a higher degree of branching of the compound according to the invention.

7 can be readily prepared in two steps from the CH-acidic compound 5. Alternatively, this compound can also be prepared from malonic acid (A. N. Blanchard, D. J. Burnell, Tetrahedron Lett. 2001, 42, 4779-4781). Such tricarboxylic triesters can also be converted into the dicarboxylic diesters by thermal decarboxylation, which means that a large number of dicarboxylic acids are very readily available.

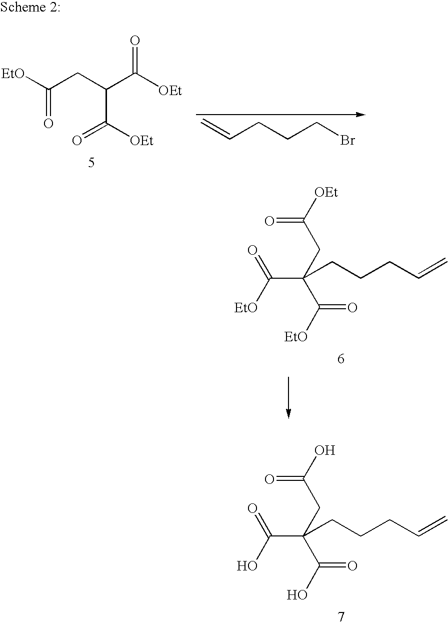

5 (200 mg; 0.81 mmol) is added, at 20-25° C., to a suspension of NaH (34 mg; 60% in oil) in a mixture of THF (3 ml) and DMF (1 ml). After approx. 10 min (evolution of hydrogen), 5-bromo-1-pentene (121 mg; 0.81 mmol) is added at 20-25° C. The resulting reaction mixture is then stirred at 50° C. for 48 hours. After it has cooled down to 20-25° C., the reaction mixture is diluted with an ammonium chloride solution (0.5 M; 2 ml). Chromatographic purification of the crude product, which is obtained by extracting with ethyl acetate, yields 6 (214 mg; 84%) as a colorless oil.

¹H-NMR (200 MHz, CDCl₃): δ=1.20-1.40 (11H); 1.93-2.10 (4H); 2.97 (s, 2H); 4.15-4.25 (OCH₂, 6H); 4.95-5.05 (2H); 5.70-5.85 (1H)

MS (ES+): m/z: 315.1 [M+H]⁺, 337.0 [M+Na]⁺; C₁₆H₂₆O₆.

NaOH (2M, 5 ml) was added, at 20-25° C., to a solution of 6 (2.0 g; 6.4 mmol) in ethanol (20 ml). This mixture was heated to 55° C. and then stirred at this temperature for 72 hours. The reaction mixture was then cooled down to 20-25° C. and the ethanol was removed in vacuo. The residue was dissolved in water/methanol (1:1, 20 ml) and loaded onto activated Dowex 50 (H⁺ form, 10 g). The product was eluted with water/MeOH (4:1, 40 ml).

Azeotropic distillation with toluene in vacuo yields 7 (1.45 g, quantitative) as a white-gray solid.

¹H-NMR (200 MHz, DMSO-d6): δ=1.15-1.29 (2H); 1.75-2.05 (4H); 2.72 (s, 2H); 4.87-5.05 (2H); 5.63-5.85 (1H).

¹³C-NMR (50 MHz, DMSO-d6): δ=23.39; 32.25; 33.41; 37.25; 54.43; 115.16; 138.33; 171.90; 172.31; 172.32.

MS (ES+): m/z: 231.0 [M+H]⁺, 253.0 [M+Na]⁺; C₁₀H₁₄O₆.

The main step in the synthesis of the compounds according to the invention is effected by means of a multicomponent reaction, with preference being given to the Ugi reaction with three (U-3CR) or four (U-4CR) components in liquid phase. In the case of the U-4CR, the amine component is reacted, in liquid phase, with the oxo component, the acid component and an isocyanate component in accordance with the following general formula:

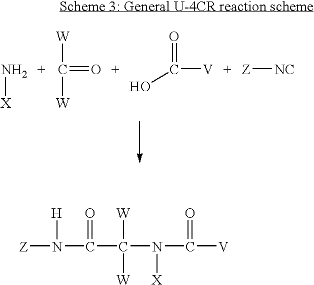

It is advantageous to use in each case one equivalent of the individual components in the reaction. It can furthermore also be advantageous to form the azomethine by means of a preliminary condensation. Aprotic, polar and nonpolar, and protic, polar solvents can be used. Protic solvents which are particularly suitable for this purpose are alcohols, such as methanol or ethanol, water or water/alcohol mixtures, and also DMF or acetonitrile. The aprotic solvents which are frequently used are dichloromethane, tetrahydrofuran or chloroform. Lewis acids, such as boron trifluoride etherate or zinc chloride, have a beneficial effect on the Ugi reaction. While the reactions are normally carried out at from −20° C. to 100° C., preference is given to reaction temperatures of between 0° C. and 50° C.

General Protocol:

A solution of the amine component (3.4 mmol) and of the oxo component (3.4 mmol) in methanol (30 ml) is stirred for 10-15 min. The isonitrile (3.4 mmol) and the acid component (3.4 mmol) are then added to this solution. The reaction solution is stirred for 12 hours. The solvent is then removed in vacuo and the crude product is purified chromatographically or by crystallization.

### Example 1

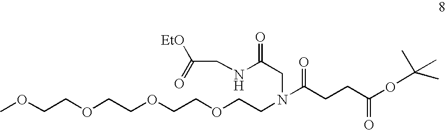

¹H-NMR (200 MHz, CDCl₃) δ=1.21 (t, 3H); 1.37 (s, 9H) 2.45-2.65 (4H); 3.32 (s, 3H) 3.45-3.65 (16H); 3.90-3.99 (2H); 4.05-4.16 (4H); 7.18 (t, NH)

MS (ES+): m/z: 507.3 [M+H]⁺, 529.3 [M+Na]⁺; C₂₃H₄₂N₂O₁₀.

### Example 2

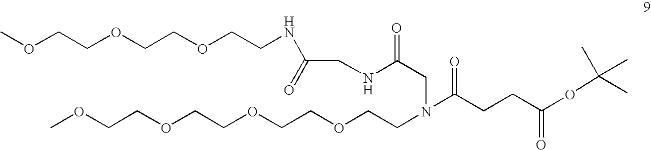

MS (ES+): m/z: 624.4 [M+H]⁺, 646.4 [M+Na]⁺; C₂₈H₅₃O₁₂

### Example 3

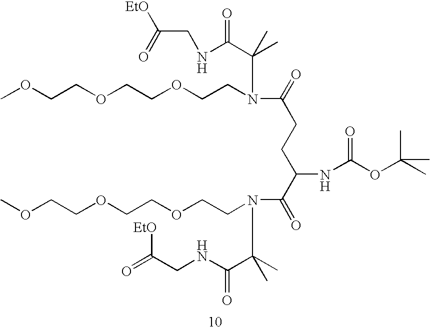

MS (ES+): m/z 902.9 [M+H]⁺; (ES−): m/z: 879.1 [M−H]⁻; C₄₀H₇₃N₅O₁₆

### Example 4

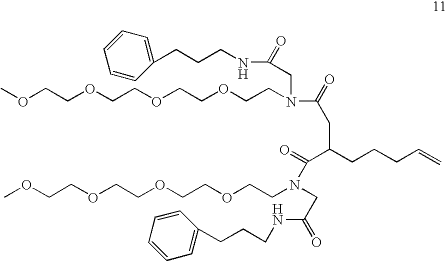

MS (ES+): m/z: 916.3 [M+H]⁺; 938.3 [M+Na]⁺; C₄₉H₇₈N₄O₁₂

It can furthermore be advantageous to use acid components which simultaneously serve as protecting group for the amino functionality. These protecting groups can subsequently be removed such that the secondary amine which is formed can also be coupled, at a later stage, to carboxylic acids using well known methods from peptide chemistry. Examples of these acids are trifluoroacetic acid and 4-pentenecarboxylic acid.

### Example 5

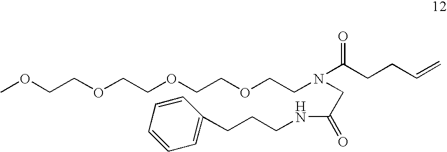

MS (ES+): m/z: 465.3 [M+H]⁺; 487.3 [M+Na]⁺; C₂₅H₄₀N₂O₆

### Example 6

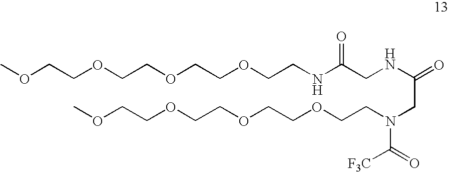

MS (ES+): m/z: 608.6 [M+H]⁺; 630.3 [M+Na]⁺; C₂₄H₄₄F₃N₃O₁₁.

In a number of cases, it can be advantageous to replace the acid component with an acid which does not react as in the Ugi reaction. Examples of acids employed are mineral acids, such as hydrochloric acid or sulfuric acid, sulfonic acids and Lewis acids, such as boron trifluoride etherate or InCl₃. In this U-3CR, water assumes the function of the acid component, with a secondary amine being formed. This secondary amine can subsequently be coupled, using a variety of amidation methods which are already known from peptide chemistry, to branched or unbranched carboxylic acid functionalities. In the case of the U-3CR, the amine component is reacted with the oxo component, the acid component (e.g. sulfuric acid) and an isocyano component in liquid phase, in accordance with the following general formula:

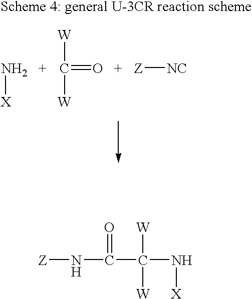

It is advantageous to in each case use one equivalent of the individual components in the reaction. It can furthermore also be advantageous to form the azomethine by means of a preliminary condensation. Aprotic, polar and nonpolar, and protic, polar solvents can be used. Protic solvents which are particularly suitable for this purpose are alcohols, such as methanol and ethanol, water or water/alcohol mixtures, as well as DMF or acetonitrile. The aprotic solvents which are frequently used are dichloromethane, tetrahydrofuran or chloroform. While the reactions are normally carried out at from −20° C. to 100° C., the reaction temperatures of between 0° C. and 50° C. are preferred.

General Protocol:

A solution of the amine component (1.2 mmol) and the oxo component (1.2 mmol) in methanol (2 ml) is stirred for 10-15 min. The isonitrile (1.2 mmol) and the acid or a Lewis acid (1.2 mmol) is then added to this solution. The reaction solution is stirred for 12 hours. The solvent is subsequently removed in vacuo and the crude product is purified chromatographically or by crystallization.

### Example 7

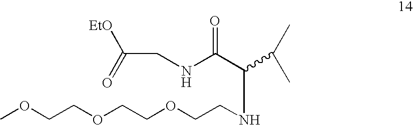

MS (ES+): m/z: 349.4 [M+H]⁺, 371.4 [M+Na]⁺; C₁₆H₃₂N₂O₆

Converting into an Active Ester Taking 7 as an Example

The tert-butyl ester is cleaved under standard conditions, e.g. using mineral acids such as HCl or HCl in dioxane. Alternatively, it is also possible to use trifluoroacetic acid.

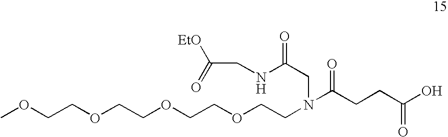

MS (ES+): m/z: 451.2 [M+H]⁺, 473.2 [M+Na]⁺; C₁₉H₃₄N₂O₁₀

16 is obtained by reacting 15 with DCC and N-hydroxy-succinimide.

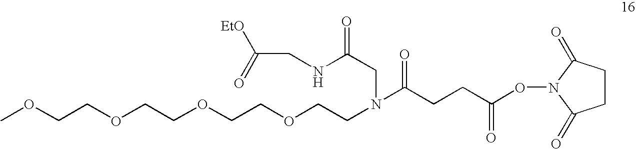

¹H-NMR (200 MHz, CDCl₃): δ=1.23 (t, 3H); 2.64-2.70 (2H); 2.82 (bs, 4H); 2.93-3.00 (2H); 3.36 (s, 3H); 3.50-3.72 (16H); 3.96-4.05 (2H); 4.08-4.20 (4H); 7.14 (t, NH)

MS (ES+): m/z: 548.3 [M+H]⁺, 570.3 [M+Na]⁺; C₂₃H₃₇N₃O₁₂

### B. EXAMPLES OF USING COMPOUNDS ACCORDING TO THE INVENTION TO MODIFY BIOPHARMACEUTICAL, PHARMACEUTICAL AND/OR SYNTHETIC ACTIVE COMPOUNDS

The following examples are intended to demonstrate the benefit of the compounds according to the invention without, however, limiting the invention.

General methods: Protein concentrations were determined in accordance with the method of Bradford using Coomassie Brilliant Blue G-250 and bovine serum albumin as the reference protein (Bradford 1976, Anal. Biochem. 72, 248-254). Denaturing polyacrylamide gel electrophoreses (SDS-PAGE) were carried out in accordance with Laemmli (1970) using 7.5% polyacrylamide gels. Proteins were then stained with Coomassie Brilliant Blue R-250. The degree of modification of lysine residues was determined, in accordance with the method of Stocks et al. (Stocks et al. 1986, Anal. Biochem. 154, 232-234), by using fluorescamine to quantify the unmodified amino groups (λₑₓ=390 nm; λₑₘ=475 nm).

Bovine serum albumin (abbreviation: BSA, Sigma), L-asparaginase (abbreviation: ASNase, ProThera), streptokinase (Sigma), trypsin (Sigma) and chymotrypsin (Sigma) were used for the experiments.

Determining the enzyme activities: L-asparaginase catalyzes the deamidation of L-asparagine to form L-aspartic acid. In order to determine the enzyme activity, ammonium which was being released in this reaction was quantified using Neβler reagent. Streptokinase activates plasminogen. Plasminogen which has been activated in this way catalyzes the hydrolysis of the tripeptide derivative D-Val-Leu-Lys-para-nitroanilide (S-2251). In order to indirectly determine the activity of streptokinase, the quantity of nitroaniline which was released was quantified photometrically at 405 nm. The para-nitroanilide derivative α-benzoylarginine-para-nitroanilide was used to determine the peptidolytic activity of trypsin, by photometrically quantifying the nitroaniline which was released at 405 nm.

Investigating the stability of the conjugates according to the invention towards proteolysis by trypsin or chymotrypsin: the conjugates, which comprise a compound according to the invention which was covalently coupled to a biopharmaceutical, pharmaceutical or synthetic active compound, were incubated at 37° C. for at least 90 min in the presence of trypsin or chymotrypsin. Aliquots were removed at different times and the residual activity of the conjugate under investigation was determined in these aliquots. Trypsin preferentially cleaves peptides and proteins C-terminally of basic amino acids (lysine and arginine residues) while chymotrypsin preferentially cleaves C-terminally of aromatic amino acids (tryptophan, phenylalanine and tyrosine residues).

### Example B1

Preparing a conjugate composed of the compound according to the invention substance 16 and L-asparaginase.

Substance 16 (0.5 eq./0.7 μl, 1 eq./1.4 μl, 2 eq./2.7 μl, 5 eq./6.8 μl, 10 eq./13.7 μl and, respectively, 20 eq./27.3 μl) dissolved in dimethyl sulfoxide (10 mg/ml) was added to 75 μl of a solution of L-asparaginase (0.5 mg/ml) in sodium carbonate buffer (pH 8.5 to 9.5) and the mixture was made up to a total volume of 150 μl using sodium carbonate buffer (pH 8.5 to 9.5). The reaction mixture was incubated at 25° C. and 300 rpm for 1 h on a thermomixer. Excess substance 16 was then removed by means of filtration in centrifuge filtration units (10 kDa cut-off) using water as rinsing liquid.

The modification only reduces the activity of the L-asparaginase to a slight extent, i.e. down to 75% residual activity when the degree of PEGylation is 41% and down to a residual activity of 60% when the degree of PEGylation is 43% (cf. Table 1). On the other hand, the PEGylation with substance 16 markedly increases the stability towards proteases (trypsin and chymotrypsin) (cf. FIGS. 1 and 2).

TABLE 1
Degree of modification, and residual activity, of the
conjugates composed of L-asparaginase and substance 16

| eqs. of 16 | | Degree of | Residual |
|---|---|---|---|
| employed | MW [Da] | modification | activity |
| 0.5 | 35544 | 3% | 100% |
| 1 | 35780 | 13% | 100% |
| 2 | 36651 | 20% | 92% |
| 5 | 37931 | 35% | 87% |
| 10 | 38798 | 41% | 75% |
| 20 | 39276 | 43% | 60% |

### Example B2

Preparing a conjugate composed of the compound according to the invention substance 16 and streptokinase.

Substance 16 (0.5 eq./0.9 μl, 1 eq./2.1 μl, 2 eq./3.9 μl, 5 eq./10.2 μl and, respectively, 10 eq./20.1 μl) dissolved in dimethyl sulfoxide (5 mg/ml) was added to 120 μl of a solution of streptokinase (0.25 mg/ml) in sodium carbonate buffer (pH 8.5 to 9.5) and the mixture was made up to a total volume of 150 μl using sodium carbonate buffer (pH 8.5 to 9.5). The reaction mixture was incubated at 25° C. and 300 rpm for 1 h on a thermomixer. Excess substance 16 was then removed by means of filtration in centrifuge filtration units (10 kDa cut-off) using water as rinsing liquid.

Steptokinase is 100% modified at the lysine residues when 10 equivalents of substance 16 are used (cf. Table 2).

TABLE 2
Degree of modification of the conjugates composed
of streptokinase and substance 16

| eqs. of 16 | | Degree of |
|---|---|---|
| employed | MW [Da] | modification |
| 0.5 | 48552 | 13% |
| 1 | 52452 | 40% |
| 2 | 55072 | 58% |
| 5 | 60366 | 96% |
| 10 | 62398 | 100% |

### Example B3

Preparing a conjugate composed of the compound according to the invention substance 16 and trypsin.

Substance 16 (0.5 eq./1.5 μl, 1 eq./2.7 μl, 2 eq./5.4 μl, 5 eq./13.8 μl and, respectively, 10 eq./27.3 μl) dissolved in dimethyl sulfoxide (10 mg/ml) was added to 120 μl of a solution of trypsin (1.0 mg/ml) in sodium carbonate buffer (pH 8.5 to 9.5) and the mixture was made up to a total volume of 150 μl using sodium carbonate buffer (pH 8.5 to 9.5). The reaction mixture was incubated at 25° C. and 300 rpm for 1 h on a thermomixer. Excess substance 16 was then removed by means of filtration in centrifuge filtration units (10 kDa cut-off) using water as rinsing liquid.

Trypsin is 44% modified at the lysine residues when using 10 equivalents of substance 16. In this connection, the residual activity increases to 137%. The increase in activity resulting from modification with polyethylene glycol-containing reagents is explained in the literature as being due to a change in the microenvironment of the active center (Zhang, Z., He, Z. & Guan, G. (1999) in Biotechnology Techniques 13: 781-786).

TABLE 3
Degree of modification, and residual activity, of the
conjugates composed of trypsin and substance 16

| eqs. of 16 | | Degree of | Residual |
|---|---|---|---|
| employed | MW [Da] | modification | activity |
| 0.5 | 28535 | 20% | 104% |
| 1 | 28891 | 25% | 109% |
| 2 | 29544 | 24% | 119% |
| 5 | 30000 | 41% | 136% |
| 10 | 30194 | 44% | 137% |

### C. OTHER EXAMPLES OF COMPOUNDS ACCORDING TO THE INVENTION AS CLAIMED IN CLAIMS 1 TO 6

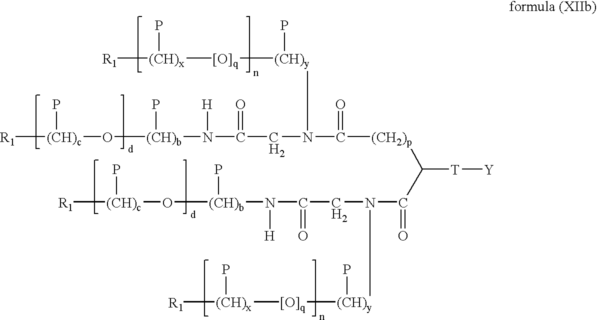

Exemplary Embodiment for Formula (XIIb):

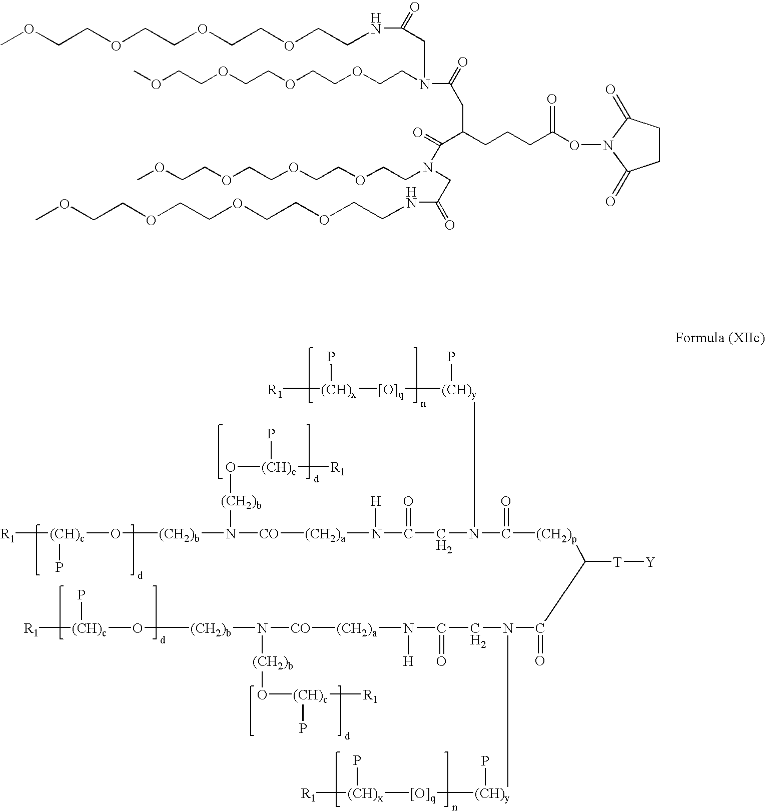

Exemplary Embodiment, Formula (XIIc): 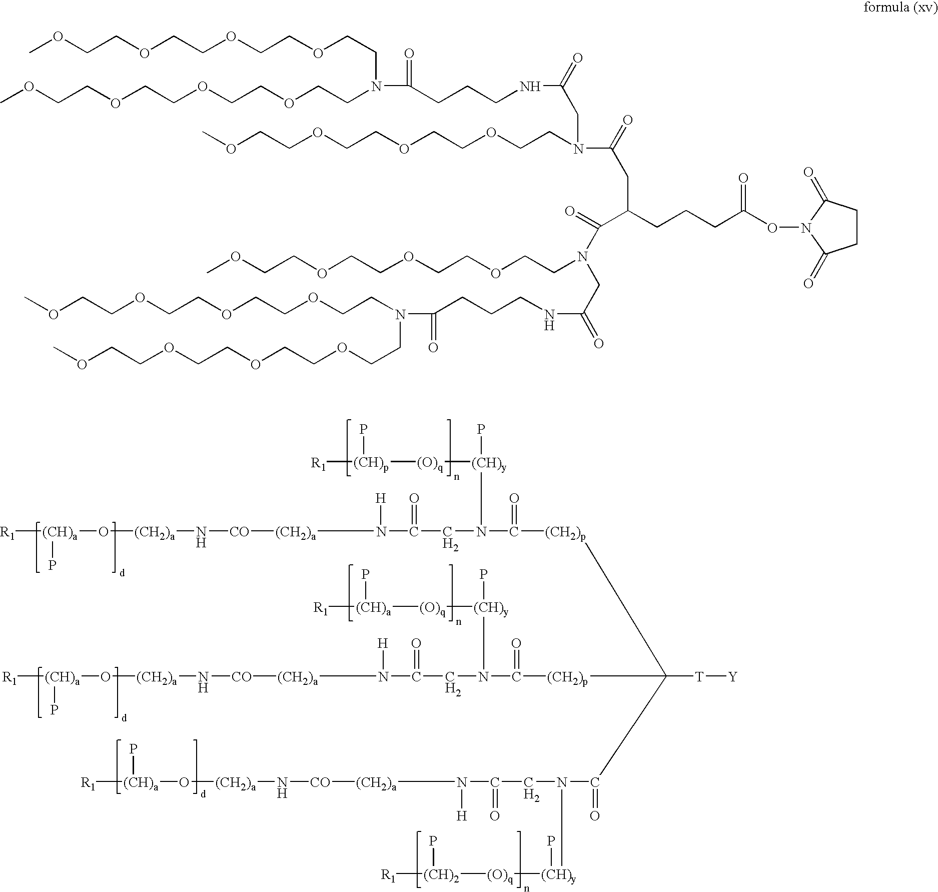
formula (XV)

Exemplary Embodiment for Formula (XV): 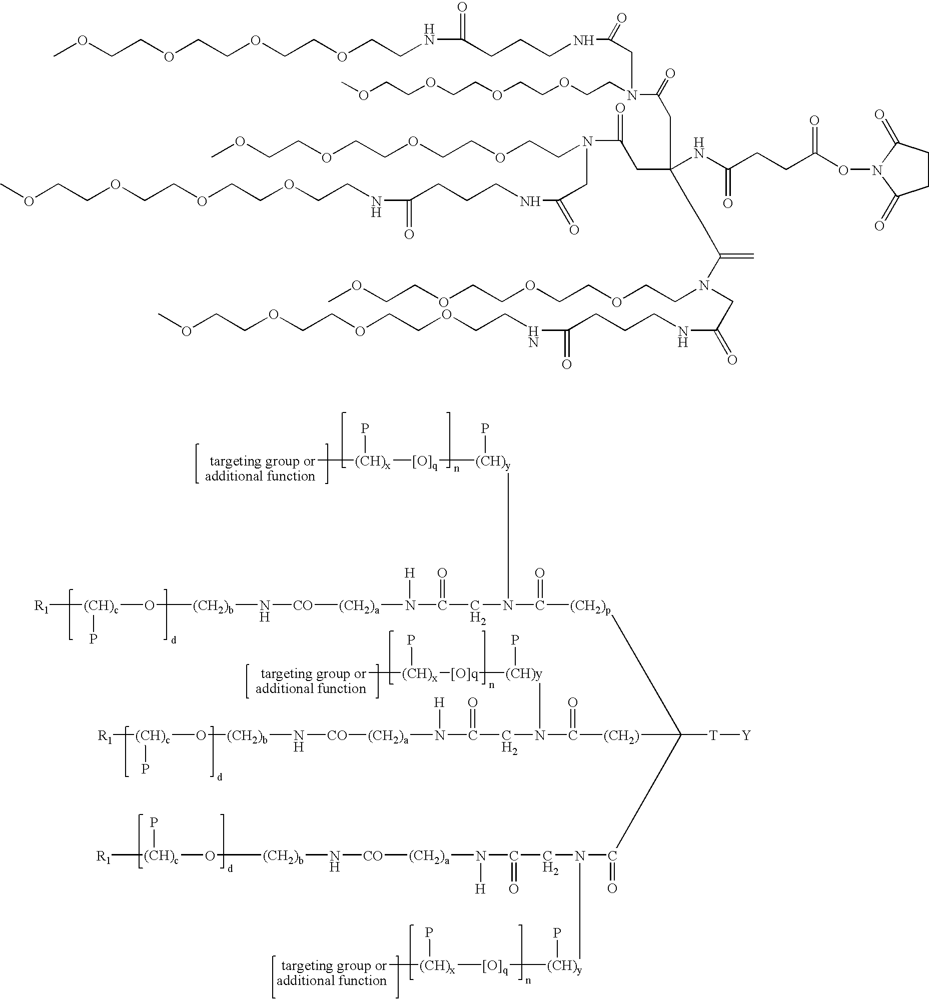
formula (XVI)

Exemplary Embodiment for Formula (XVI):

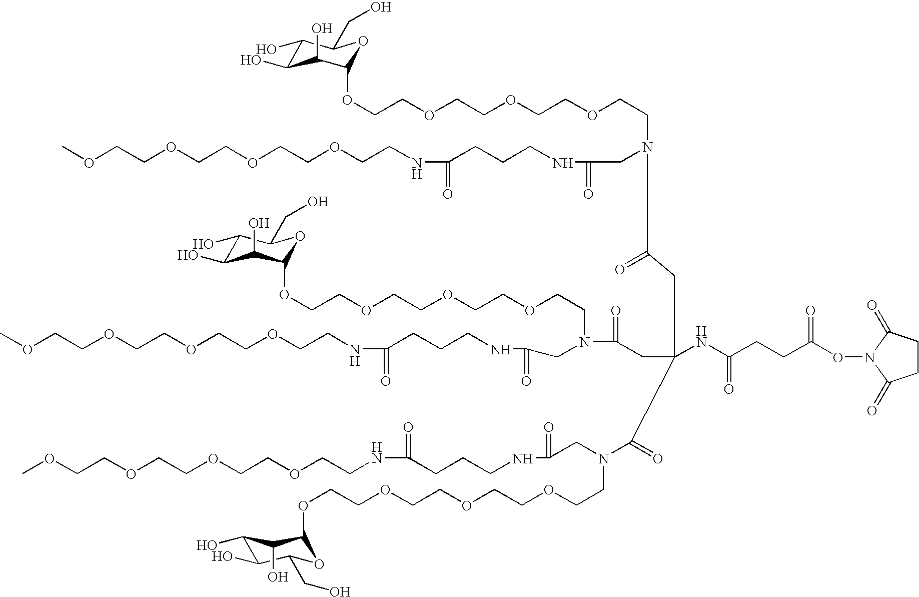

In Examples C (formulae XIIb, XIIc, XV and XVI):

P is, on each occasion independently, H, OH, C₁-C₄-alkyl, O—R₂ or CO—R₃,

R₁ is H, OH or a hydrocarbon residue which possesses from 1 to 50 carbon atoms and which can contain heteroatoms, in particular O and/or N,

R₂ is, on each occasion independently, a hydrocarbon residue having from 1 to 6 C atoms,

R₃ is OH or NR₄R₅,

R₄ and R₅ are, in each case independently, H or a hydrocarbon residue which can contain heteroatoms, in particular O and/or N, where R₄ and R₅ can also together form a ring system,

d and n are, on each occasion independently, an integer of from 1 to 1000,

c and x are, on each occasion independently, an integer of from 1 to 10, and

a, b, p and y are, independently, an integer of from 0 to 50, and

q is, on each occasion independently, 0 or 1.

1. A compound of the formula [symbol] 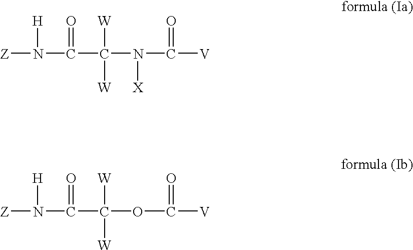
in which
the residues V, W, X and Z are in each case, independently of each other, a hydrocarbon residue which can contain heteroatoms and/or V, W and/or X is/are hydrogen, characterized in that at least one of the residues V, W, X and/or Z carries a binding group Y and in that the residues V, W, X and Z together exhibit at least one group of the formula (II) 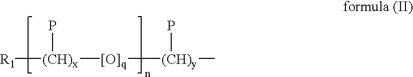
in which
P is, on each occasion independently, H, OH, O—R₂
or CO—R₃,
R₁ is H or a hydrocarbon residue which has from 1 to 50 carbon atoms and which can contain heteroatoms,
R₂ is, on each occasion independently, a hydrocarbon residue having from 1 to 6 C atoms,
R₃ is OH or NR₄R₅,
R₄ and R₅ are, in each case independently, H or a hydrocarbon residue which can contain heteroatoms, where R₄ and R₅ can also together form a ring system,
n is, on each occasion independently, an integer of from 3 to 1000, and
x is, on each occasion, an integer of from 1 to 10, and
y is an integer of from 0 to 50, and
q is, on each occasion, 1.

2. A compound as claimed in claim 1, characterized in that the binding group Y is selected from groups which are able to bind to an amino group, a thiol group, a carboxyl group, a guanidine group, a carbonyl group, a hydroxyl group, a heterocycle, a C-nucleophilic group, a C-electrophilic group, a phosphate or a sulfate, or are able to form a chelate or a complex with metals or are able to bond to silicon-containing surfaces.

3. A compound as claimed in claims 1, characterized in that it contains at least three groups of the formula (II).

4. A compound as claimed in claim 1, characterized in that at least one of the residues X and/or Z is branched and contains at least two groups of the formula (II).

5. A compound as claimed in claim 1, wherein at least one of the residues X and/or Z additionally possesses a targeting group.

6. A compound having the formula (XIV) 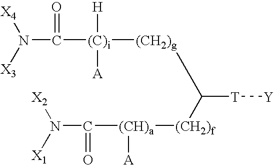
in which
h and i are, on each occasion independently, 0 or 1,
g and f are, on each occasion independently, an integer between 0 and 10, preferably between 0 and 5,
A is, on each occasion, H or —(CO)—NX₂, and
X₁, X₂, X₃ and X₄, and also X, have, in each case independently of each other, the meanings given above for X, where the compound exhibits at least two groups of the formula (II) 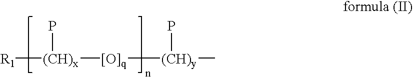
in which
P is, on each occasion independently, H, OH, O—R₂ or CO—R₃,
R₁ is H or a hydrocarbon residue which has from 1 to 50 carbon atoms and which can contain 5 heteroatoms,
R₂ is, on each occasion independently, a hydrocarbon residue having from 1 to 6 C atoms,
R₃ is OH or NR₄R₅,
R₄ and R₅ are, in each case independently, H or a hydrocarbon residue which can contain heteroatoms,
where R₄ and R₅ can also together form a ring system,
n is, on each occasion independently, an integer of from 3 to 1000 and
x is, on each occasion, an integer of from 1 to 10, and
y is an integer of from 0 to 50, and
q is, on each occasion, 1.

7. A method for preparing a compound as claimed in claim 1, characterized in that the compounds of the formulae
X′—NH₂ (IV)
(W′)₂C═O (V)
Z′—NC (VI),
and
V′—COOH (VII)
are reacted with each other, as starting compounds, in a multicomponent reaction, where V′, W′, X′ and Z′ are, in each case independently of each other, a hydrocarbon residue which can optionally contain heteroatoms and/or V′, W′ and/or X′ are hydrogen, where at least one of the residues V′, W′, X′ and Z′ carries a binding group Y and where the residues V′, W′, X′ and Z′ together possess at least two groups of the formula (II) 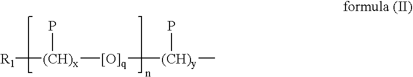
in which
P is, on each occasion independently, H, OH, O—R₂ or CO—R₃,
R₁ is H or a hydrocarbon residue which has from 1 to 50 carbon atoms and which can contain heteroatoms,
R₂ is, on each occasion independently, a hydrocarbon residue having from 1 to 6 C atoms,
R₃ is OH or NR₄R₅,
R₄ and R₅ are, in each case independently, H or a hydrocarbon residue which can contain heteroatoms,
where R₄ and R₅ can together also form a ring system,
n is, on each occasion independently, an integer of from 3 to 1000, and
x is, on each occasion, an integer of from 1 to 10, and
y is an integer of from 0 to 50, and
q is, on each occasion, 1.

8. The method as claimed in claim 7, characterized in that at least one of the residues V′, W′, X′ and/or Z′ contains at least one further functionality selected from NH₂, C═O, NC and/or COOH.

9. A conjugate which comprises a compound of the formula (I), as defined in claim 1, which is covalently bonded to a biopharmaceutical, pharmaceutical and/or synthetic active compound.

10. A conjugate which comprises a compound of the formula (I), as defined in claim 1, which is covalently bonded to a surface and/or a biocatalyst.

11. A conjugate which comprises a compound of the formula (I), as defined in claim 1, which is covalently bonded to an enzyme.

12. A conjugate which comprises a compound of the formula (I), as defined in claim 1, which is covalently bonded to medicinal products or adjuvants for administering active compounds.

13. A pharmaceutical composition which comprises a compound as claimed in claim 1.

14. A diagnostic composition which comprises a compound as claimed in claim 1.

15. A pharmaceutical for treating cancer or coronary diseases, metabolic diseases, neuronal or cerebral diseases, e.g. Alzheimer's or Parkinson's, or inflammatory processes, e.g. infections, and immune or autoimmune diseases, in particular rheumatoid arthritis, comprising the conjugate as claimed in claim 9.

16. A method for preparing a substance library, characterized in that at least two different compounds as claimed in claim 1 are prepared using the method as claimed in claim 7 or 8.

17. A substance library which comprises at least two different compounds of the formula (I), as defined in claim 1.

18. A kit which comprises
(a) at least one compound as claimed in one of claims 1 to 6 and also
(b) buffer solutions and, where appropriate,
(c) standard proteins and/or means for purifying conjugates which have been formed together with the compound from (a).

19. A pharmaceutical composition comprising the conjugate as claimed in claim 9.

20. A diagnostic composition comprising the conjugate as claimed in claim 9.
